(19) United States
(12) Patent Application Publication
(10) Pub. No.: US 2024/0189819 A1
(43) Pub. Date: Jun. 13, 2024

(54) Microfluidic Device for Analyzing Sample Material, and Method for Operating a Microfluidic Device

(71) Applicant: Robert Bosch GmbH, Stuttgart (DE)

(72) Inventor: Daniel Sebastian Podbiel, Rutesheim (DE)

(21) Appl. No.: 18/554,615

(22) PCT Filed: Apr. 5, 2022
(86) PCT No.: PCT/EP22/58943

§ 371 Date: Oct. 9, 2023

(30) Foreign Application Priority Data
Apr. 13, 2021 (DE) 10 2021 203 638.4

(51) Int. Cl. B01L 3/00

(52) U.S. Cl. CPC B01L 3/502753; B01L 3/502707; B01L 3/502715; B01L 3/50273; B01L 3/502738; B01L 2200/027; B01L 2200/028; B01L 2200/0621; B01L 2200/10; B01L 2200/16; B01L 2300/0681; B01L 2300/0816; B01L 2300/087; B01L 2400/0487; B01L 2400/0605

(57) ABSTRACT

The disclosure relates to a microfluidic device for analyzing sample material, said device comprising a microfluidic network that includes a first network portion for purifying sample material as well as a second network portion for amplifying sample material, the second network portion being connected to the first network portion via a connection duct, the first and second network portions being separable from one another.

### THE PRIOR ART

The invention proceeds from a microfluidic device for analyzing sample liquid and a method for operating a microfluidic device belonging to the class of patent specified in the independent claim. The object of the present invention is also a computer program.

Microfluidic analysis systems, which are referred to lab-on-chips (abbreviated as LoCs), enable the automated, reliable, fast, compact, and cost-effective processing of patient samples for medical diagnostics. By combining a plurality of operations for controlled manipulation of fluids, complex molecular diagnostic test procedures can be performed in a lab-on-chip cartridge, which can also be referred to as a microfluidic analysis device. Lab-on-chip cartridges can, e.g., be produced cost-efficiently from polymers using series production processes such as injection molding, punching or laser transmission welding. The processing of a lab-on-chip cartridge is performed in an associated analysis device. Various types of microfluidic analysis systems are known.

### DISCLOSURE OF THE INVENTION

Against this background, the approach presented herein provides a microfluidic device for analyzing sample material and a method for operating a microfluidic device and furthermore a control device using this method, and finally a corresponding computer program according to the independent claims. Advantageous embodiments of and improvements to the device specified in the independent claim are made possible by the measures presented in the dependent claims.

In general, a pressure-based microfluidic analysis device comprises a variety of active microfluidic elements, such as valves and pump chambers, which are interconnected in a suitable network of microfluidic ducts. The pneumatic control of the active microfluidic elements in turn is performed via pneumatic microducts within the microfluidic device, which can be pressurized or depressurized via an interface of the microfluidic analysis device, which is designed to be as compact as possible, to an analysis device, which can also function as a processing unit, in order to induce an actuation of the microfluidic elements by a corresponding deflection of an elastic membrane. In the design of such a microfluidic analysis device, the question therefore arises, in particular, of a particularly advantageous arrangement of the elements and configuration of the microfluidic network in order to be able to address a predetermined range of applications with the microfluidic device in a particularly advantageous manner. The microfluidic device presented herein provides a particularly advantageous configuration of a microfluidic network with active pneumatically controllable elements for processing a sample liquid. Advantageously, the consumption of reagents required to perform a test run can be minimized, whereby at the same time particularly high reliability in microfluidic processing can be achieved.

A microfluidic device for analyzing sample material is presented, said device comprising a microfluidic network that includes a first network portion for extracting and thereby purifying sample material, as well as a second network portion for reproducing and thereby amplifying sample material, the second network portion being connected to the first network portion via a connection duct, the first and second network portions being separable from one another.

The microfluidic device can, e.g., be a lab-on-chip cartridge as part of a pressure based microfluidic analysis system. Liquid transport within the microfluidic analysis device can take place by applying, mostly pneumatic, pressure. The sample material, which can, e.g., originate from a sample substance added to the microfluidic device and processed within the microfluidic device, can, e.g., be a sample liquid. A sample substance can be understood as the sample which can be added into the cartridge, e.g. a liquid sample or a swab sample. In contrast, the sample material can in particular also only be components of the sample substance that were obtained from the sample substance, e.g. by extraction. For example, this sample liquid can be an aqueous solution, e.g., obtained from a biological substance, e.g. of human origin, such as a body fluid, a smear, a secretion, sputum, a tissue sample, or a device with attached sample material. The sample liquid can contain, e.g., species of medical, clinical, diagnostic, or therapeutic relevance, such as bacteria, viruses, cells, circulating tumor cells, cell-free DNA, proteins or other biomarkers or, in particular, constituents from the objects mentioned. For example, the sample liquid can be a master mix or components thereof, e.g., for performing at least one amplification reaction in the microfluidic analysis device, e.g., for molecular level DNA verification, e.g. an isothermal amplification reaction or polymerase chain reaction. For example, sample material can first be extracted in the device from an added sample substance, e.g. a liquid sample or swab sample. That is to say, sample material can be purified to enable subsequent amplification of components of the sample substance. The purification is particularly advantageous as the original sample substance can contain components, so-called inhibitors, which would adversely affect the amplification reaction. In the subsequent amplification reaction, only components of the original sample substance, such as certain DNA base sequences, can be amplified, that is, reproduced. For example, by applying positive or negative pressure locally, an elastic membrane as a component of the microfluidic device can be selectively deflected into recesses in the microfluidic device in order to draw liquid into the recesses or displace it from the recesses in this way. Due to the elastic membrane, a separation between the microfluidic, liquid-conducting areas of the device on the one hand, as well as the pneumatic areas and the external environment on the other hand, can be achieved at the same time. For example, depending on the size of a recess in a liquid-conducting region and the associated displacement volume when the membrane is deflected into that region, there can be a microfluidic pump chamber with a large displacement volume for ostensibly generating a flow in the device or a microfluidic valve with small displacement volume for ostensibly controlling the flow in the device. By a combination of a total of at least three microfluidic pump chambers and additionally or alternatively valves, a directed flow in the microfluidic device can be produced by successive actuation of the elements. For example, a pump chamber can be combined with two circumferential valves, that is, an inlet valve and an outlet valve, to achieve directed fluid transport. Moreover, for example, three similar elements with comparable displacement volumes can also be combined with each another to cause peristaltic fluid transport by successive actuations in series.

The device presented herein advantageously features a differentiation of the various functionalities that can be provided by the microfluidic device in the form of microfluidically separable network portions. The term “network portion” can in this context be understood to mean a plurality of interconnected microfluidic elements. As mentioned hereinabove, the microfluidic elements can be passive elements, e.g. chambers or actuatable elements, e.g. pump chambers, pumps, or valves. Preferably, a network portion comprises at least two, preferably more than two, fluidically connected microfluidic elements. The phrase “two separable network portions” is in particular understood to mean that the two network portions are arranged in two regions separated from each other locally and, in a preferable embodiment, can be separated from each other by a planar cut. Alternatively or additionally, two separable network portions can be understood to mean that the two network portion systems are connected to one another by one or more ducts, preferably only by one duct, whereby the ducts, or the duct, can preferably be fluidically separated from each another by at least one fluidic separating element, e.g., a valve.

Due to the separable network portions, multiple control of microfluidic elements can be advantageously enabled, which can be associated with different network portions. Furthermore, a separation of the performance of purification and amplification in separate network portions enables a reduction in the number of rinsing steps as well as a suppression of an undesired microfluidic crosstalk between different sequence steps.

According to one embodiment, the first network portion and the second network portion can be arranged in a linear topology. In particular, the term “linear topology” can be understood to mean that the network portions are linear or arranged in a row. Due to the linear network topology, dead volumes which can occur when performing a test run within the microfluidic analysis device can be particularly low. In this advantageous manner, the amount of reagents required to perform the test run within the microfluidic device can be reduced.

According to a further embodiment, the first network portion and additionally or alternatively the second network portion can comprise a plurality of fluidically separable functional modules. For example, each functional module can perform a particular task within a purification or analysis process and can have a variable number of individual elements such as valves, pump chambers or storage chambers for this purpose. The individual functional modules can be separable from each other by means of, e.g., isolating valves. Furthermore, at least some of the functional modules can be arranged in a linear topology in a preferable embodiment. The functional modules can, e.g., be successively controlled during a purification and analysis process to, e.g., first extract a species from a sample substance and then enable verification of the species or components thereof in separate regions of the microfluidic analysis device presented. Advantageously, this can prevent possible crosstalk, i.e., undesirable mixing of different liquid solutions, which can typically occur due to multiple use of certain regions of the microfluidic network. In this way, the efficiency of chemical reactions that occur within the microfluidic analysis device can be increased and the sensitivity which can be achieved in a verification process can be increased.

According to a further embodiment, the device can comprise a control connection for applying pressure, whereby the control connection can be connected by a first control duct to an element of a functional module of the first network portion and by a second control duct to an element of a functional module of the second network portion. The element can, e.g., be a valve or pump chamber or other microfluidic element of the device. For example, a positive or negative pressure can be applied to the control connection, which can also be referred to as a port or control port, to control the elements of the functional modules of the network portions. In this advantageous manner, a plurality of active elements, which each belong to different functional modules, can be switched via a common control duct. In other words, multiplexing is therefore possible during control. Multiplexing in the control of the active microfluidic elements can advantageously reduce the number of control ports needed to control a predetermined microfluidic network. In this way, the costs incurred for the analysis device, in particular the processing unit, can be reduced. In addition, multiplexing in the control of the active microfluidic elements can be used to realize a microfluidic network with an increased range of functions for a predetermined number of control ports. In this way, the range of application addressed by such a microfluidic analysis device can be extended.

According to a further embodiment, the first network portion can comprise a first functional module for providing liquid reagents and additionally or alternatively a second functional module for adding a sample substance and additionally or alternatively a third functional module for filtering sample material and additionally or alternatively a fourth functional module for storing liquids and additionally or alternatively a pump module for establishing a fluid transport between the functional modules and additionally or alternatively the second subnetwork comprises at least a first functional module for amplifying sample material and additionally or alternatively a second functional module for providing at least one amplification reaction bead. For example, the functional modules of the first network portion can be employed successively to purify or extract sample material. The first functional module can, e.g., provide liquid reagents in which, e.g., sample material can be dissolved and additionally or alternatively transported within the device. In the second functional module, a sample substance can be added, e.g. in the form of a sample liquid or a swab sample, and, if necessary, released from a carrier element, e.g. a sampling device. Using the third functional module, certain species can, e.g., be filtered out of the sample substance such that only the portion of the sample material to be analyzed can be transported further during the process. In the fourth functional module, liquid that accumulates during the purification of the sample substance can, e.g., be stored or temporarily retained. By means of the pump module, a liquid transport between the various functional modules and additionally or alternatively between the network portions can be established. For example, after purifying the sample substance in the functional modules of the first network portion, a fluid enriched with sample material can then be transported through the connection duct from the first network portion to the second network portion. Sample material can in this case be, e.g., reproduced or amplified in the first functional module, while what is referred to as an amplification reaction bead can, e.g., be provided in a second functional module. Advantageously, the number of rinsing steps required to perform a test run within the microfluidic device can thus be reduced. In this way, the amount of time required to perform a test run can be reduced.

According to a further embodiment, the fourth functional module of the first network portion and the first functional module of the second network portion can be fluidically connected by the connection duct. For example, liquid accumulating during purification of the sample substance can be stored in the fourth functional module of the first network portion. Using the connection duct, a liquid with sample material can be transferred to the first functional module of the second network portion and sample material can subsequently be amplified. Advantageously, only connecting the two functional modules can simplify the separation of individual functional modules as well as the network portions from each other, and the occurrence of cross-contamination between different process steps, that is to say undesirable mixing of different liquid solutions used during the execution of the test run, can be prevented.

According to a further embodiment, the fourth functional module of the first network portion can comprise a first shutoff valve for closing the connection duct, and additionally or alternatively, the first functional module of the second network portion can comprise a second shutoff valve for closing the connection duct. For example, the first shutoff valve can be kept closed while sample material is processed within the first network portion. This has the advantage that an undesirable premature intrusion of a transfer fluid with sample material used in the first network portion into the second network portion can be prevented. Similarly, after transport into the second network portion, where both shutoff valves can be open, closing the second shutoff valve can advantageously prevent backflow of the transfer fluid with the sample material into the first network portion.

According to a further embodiment, the device can comprise a valve control connection for applying a pressure, whereby the first shutoff valve and the second shutoff valve can be controlled by means of the valve control connection. For example, the first shutoff valve and the second shutoff valve can be simultaneously closed or opened by means of the valve control connection to enable or prevent fluid transfer between the first network portion and the second network portion of the device. Advantageously, the common control can reduce the number of control elements required for processing the device, thus reducing the cost of the system.

According to a further embodiment, the fourth functional module of the first network portion can be designed to output sample material. For example, if only purification and extraction of sample material is performed in the device, the sample material can be removed from the microfluidic device after performing an extraction, for example, without transferring it to the second network portion. For this purpose, the fourth functional module can, e.g., comprise a output means used to output the sample material. Advantageously, the device can thereby be used variably for different analysis processes.

According to a further embodiment, the first network portion can comprise an additional second functional module for adding a sample substance. For example, multiple sample input chambers can be implemented by the linear network topology of the device, whereby they can be used in an equivalent manner for subsequent microfluidic processing of the sample substance added into at least one of the sample input chambers in the microfluidic network of the device. A first sample input chamber can in this case be designed, e.g., specifically for adding a liquid sample in an optimized form, while a second sample input chamber can be designed specifically, e.g., for adding a swab sample, that is to say a sampling device with attached sample material. In this advantageous manner, the microfluidic analysis device can be employed in a universal manner for an examination of different sample substances.

According to a further embodiment, the device can comprise at least one further control connection for applying pressure, whereby the further control connection can be connected by a further control duct either to an element of a functional module of the first network portion or to an element of a functional module of the second network portion. The microfluidic device can, e.g., comprise a plurality of control connections, whereby some control connections can be designed to control multiple elements of various functional modules, while other control connections can be used to control a particular element of a functional module. Advantageously, such a precise control of all elements of the functional modules is possible.

Further presented is a method for operating a variant of the microfluidic device previously introduced, whereby the method comprises a step of extracting sample material using the first network portion, a step of transferring sample material from the first network portion into the second network portion, and a step of amplifying sample material using the second network portion.

According to one embodiment, the step of extraction can comprise a substep of mixing and additionally or alternatively a substep of dissolving and additionally or alternatively a substep of lysis and additional or alternatively a substep of filtering and additionally or alternatively a substep of rinsing. Additionally or alternatively, the step of transfer can comprise a substep of elution and additionally or alternatively, the step of amplification can comprise a substep of dissolving and additionally or alternatively a substep of reproduction and additionally or alternatively a substep of acquisition.

This method can, e.g., be implemented as software or hardware, or in a mixed form of software and hardware, e.g. in a control unit.

The approach presented herein also provides a control unit designed to perform, control, or implement the steps of a variant of a method presented herein in corresponding devices. This embodiment of the invention in the form of a control unit can also quickly and efficiently achieve the problem underlying the invention.

For this purpose, the control unit can comprise at least one computing unit for treating signals or data, at least one storage unit for storing signals or data, at least one interface to a sensor or an actuator for reading sensor signals from the sensor or for outputting control signals to the actuator, and/or at least one communication interface for reading or outputting data embedded in a communication protocol. The computing unit can, e.g., be a signal processor, a microcontroller or the like, whereby the memory unit can be a flash memory, an EEPROM, or a magnetic memory unit. The communication interface can be designed to read in or output data wirelessly and/or by wire, whereby a communication interface capable of reading or outputting data transmitted by wire can read said data, e.g. electrically or optically, from a corresponding data transmission line or output the data to a corresponding data transmission line.

The term “control unit” can in the present context be understood as an electrical device that processes sensor signals and outputs control signals and/or data signals as a function thereof. The control unit can comprise an interface, which can be designed as hardware and/or software. Given a hardware design, the interfaces can, e.g., be part of what is referred to as an ASIC system, which contains various functions of the control unit. However, it is also possible that the interfaces are separate, integrated circuits or at least partially consist of discrete structural elements. Given a software design, the interfaces can be software modules provided on, e.g., a microcontroller in addition to other software modules.

A computer program product or a computer program with program code that can be stored on a machine-readable carrier or storage medium, e.g., a semiconductor memory, a hard disk memory, or an optical memory, and that is used to perform, implement, and/or control the method steps according to one of the embodiments described above is advantageous as well, in particular when the program product or program is executed on a computer or device.

Exemplary embodiments of the approach presented herein are shown in the drawings and explained in greater detail in the following description. Shown are:

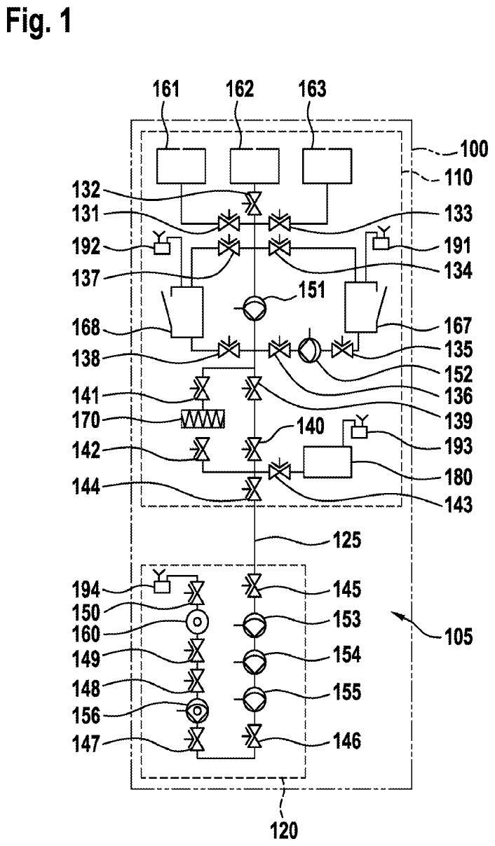

FIG. 1 a schematic view of an exemplary embodiment of a microfluidic device;

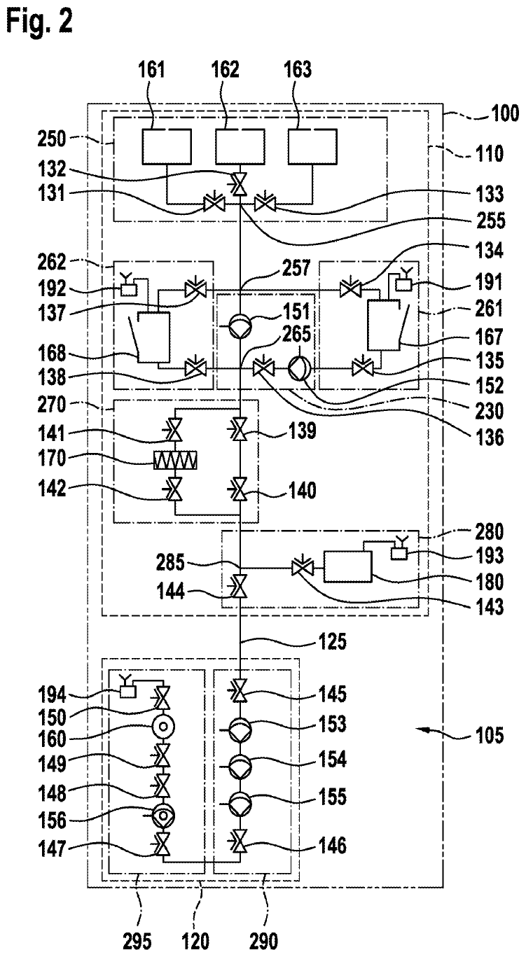

FIG. 2 a schematic view of an exemplary embodiment of a microfluidic device;

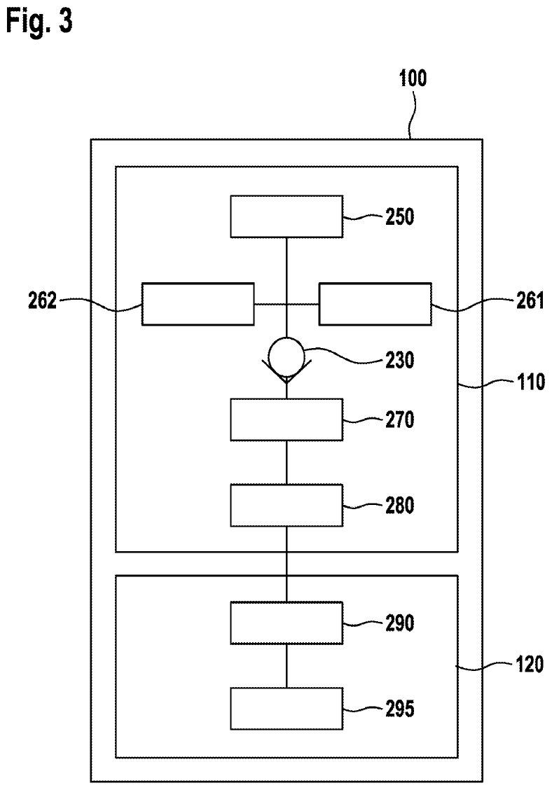

FIG. 3 a schematic view of an exemplary embodiment of a microfluidic device;

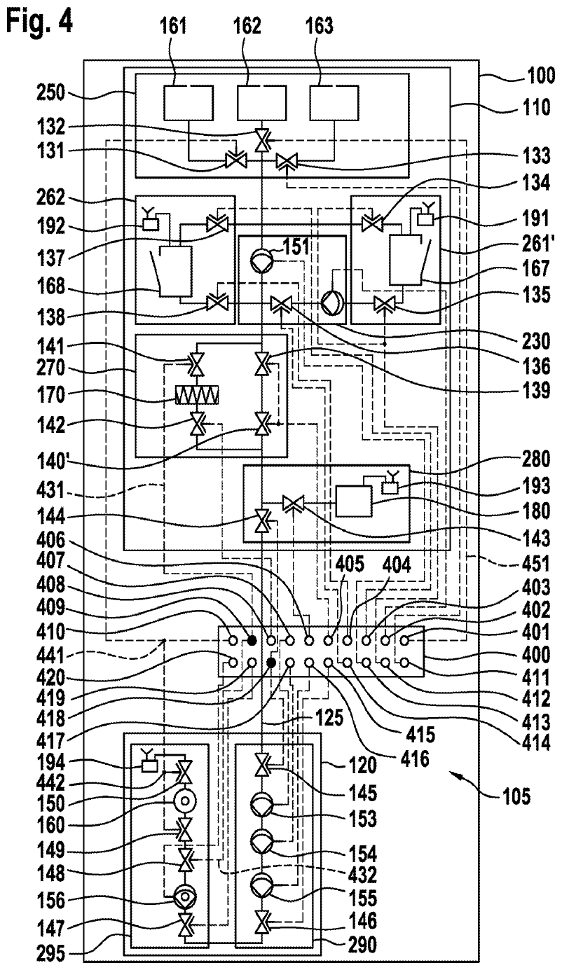

FIG. 4 a schematic view of an exemplary embodiment of a microfluidic device;

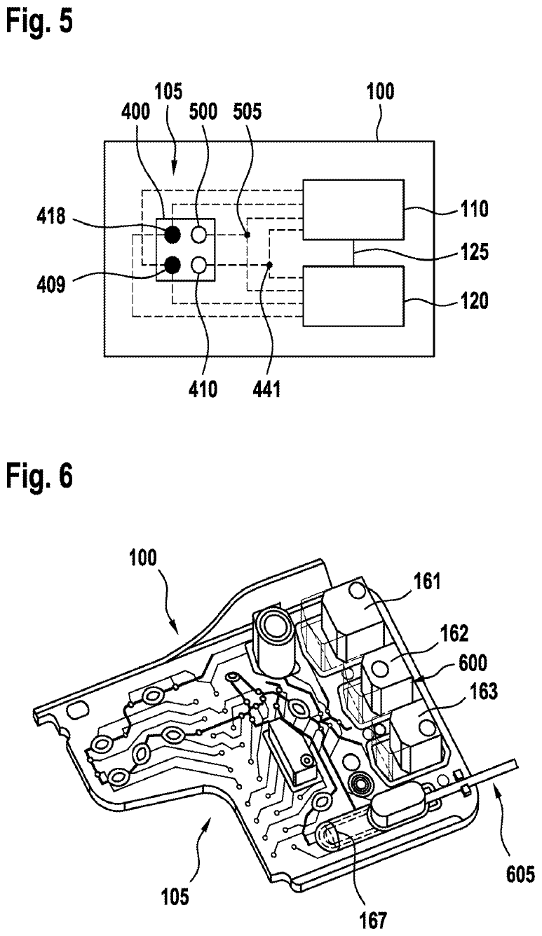

FIG. 5 a schematic view of an exemplary embodiment of a microfluidic device;

FIG. 6 a perspective top view of an exemplary embodiment of a microfluidic device;

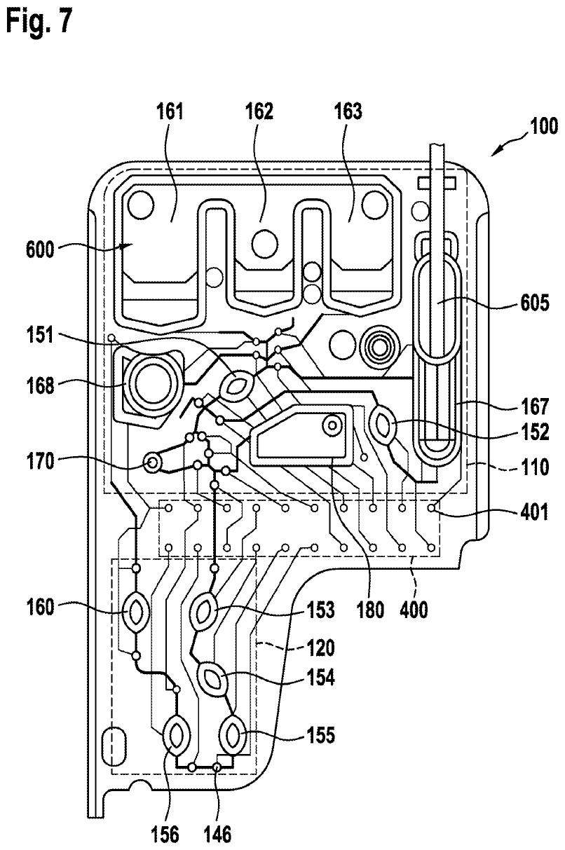

FIG. 7 a top view of an exemplary embodiment of a microfluidic device;

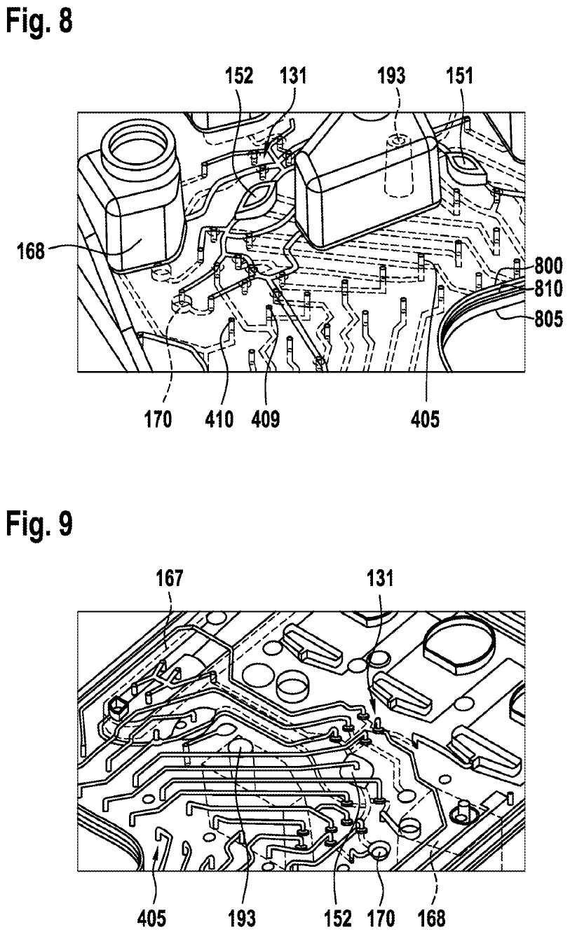

FIG. 8 a perspective top view of a section of the microfluidic device according to an exemplary embodiment;

FIG. 9 a perspective bottom view of a section of the microfluidic device according to an exemplary embodiment;

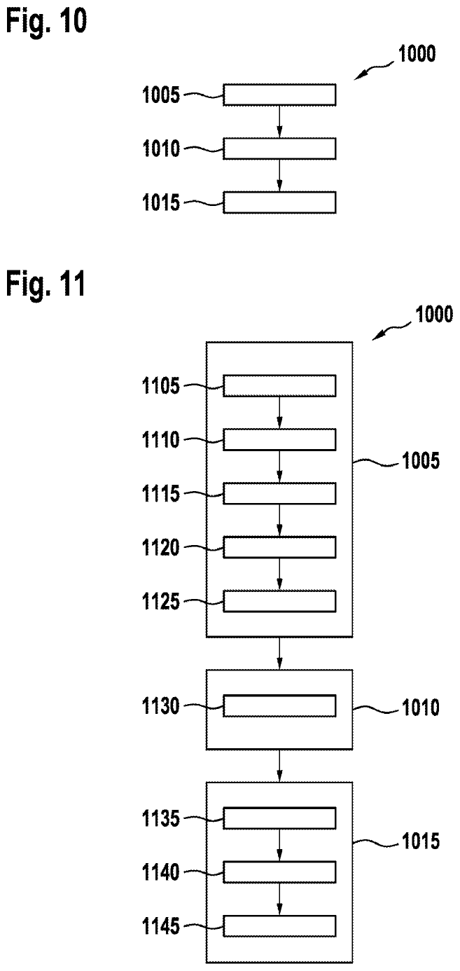

FIG. 10 a flow chart of a method 1000 for operating a microfluidic device according to an exemplary embodiment;

FIG. 11 a flow chart of a method 1000 for operating a microfluidic device according to an exemplary embodiment;

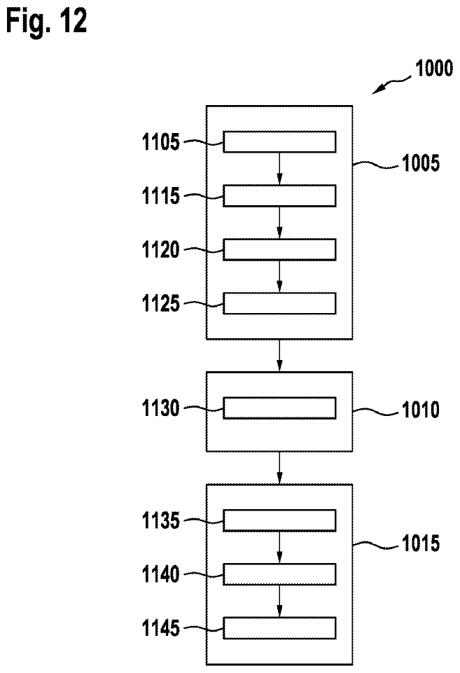

FIG. 12 a flow chart of a method 1000 for operating a microfluidic device according to an exemplary embodiment;

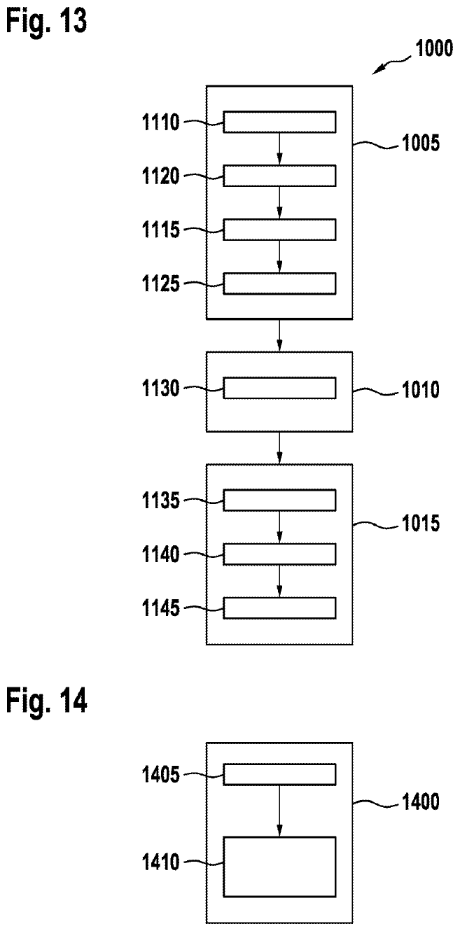

FIG. 13 a flow chart of a method 1000 for operating a microfluidic device according to an exemplary embodiment; and

FIG. 14 a schematic view of an exemplary embodiment of an analysis device for receiving a microfluidic device.

In the following description of advantageous embodiments of the present invention, identical or similar reference characters are used for the elements shown in the various drawings and acting similarly, whereby a repeated description of these elements has been omitted.

FIG. 1 shows a schematic view of an exemplary embodiment of a microfluidic device 100. The device 100 in this exemplary embodiment comprises a microfluidic network 105 having a linear topology. The network 105 is divided into a first network portion 110 and a second network portion 120, whereby both network portions 110, 120 are marked by dashed lines in the illustration shown here. The two network portions 110, 120 are connected to each other via a microfluidic connection duct 125. Both the first network portion 110 and the second network portion 120 in this case comprise further microfluidic ducts that are shown as solid lines in the figure shown here in a schematic manner that connect different microfluidic elements to each other. In this exemplary embodiment, the network 105 of the microfluidic device 100 is in particular characterized by 20 active microfluidic valves 131, 132, 133, 134, 135, 136, 137, 138, 139, 140, 141, 142, 143, 144, 145, 146, 147, 148, 149, 150. Only by way of example, two valves 144, 145 are arranged on the connection duct 125 and designed as a first shutoff valve 144 and a second shutoff valve 145 for closing the connection duct. The network portions 110, 120 are therefore separable from one another. The network 105 also comprises six active microfluidic pump chambers 151, 152, 153, 154, 155, 156, a passive microfluidic chamber 160, three pre-storage chambers with deaeration openings 161, 162, 163 for long-term stable pre-storage of liquid reagents, two sample input chambers 167, 168, which enable sample input, a filter element 170, which enables an extraction of components from a liquid, a liquid storage chamber 180, which serves to hold liquids after processing in the microfluidic network 105, as well as four deaeration devices 191, 192, 193, 194, which are used to deaerate the microfluidic network. In this exemplary embodiment, the total of 26 active microfluidic elements can be controlled by pneumatic control ducts, as illustrated by the dashes branching perpendicular to the microfluidic ducts in the reference characters. The arrangement and linkage of the specified elements forms the microfluidic network 105, which only has a linear topology (by way of example).

The microfluidic network 105 of the microfluidic device 100 comprises the first network portion 110, which is provided for the purification of sample material, and the second network portion 120, which is provided for the amplification of sample material. The two network portions 110, 120 are fluidically connected to each other via a microfluidic connection duct 125. As a result, microfluidic processing, within the context of the execution of a test run, can be performed successively, that is to say one after the other, within the two network portions 110, 120. Accordingly, a combination of active microfluidic elements, which are each arranged in different network portions 110, 120, can optionally be controlled by a common control duct.

FIG. 2 shows a schematic view of an exemplary embodiment of a microfluidic device 100. The device 100 shown in this case corresponds to or resembles the device described in the preceding figure and comprises a microfluidic network 105 divided into a first network portion 110 and a second network portion 120. In this exemplary embodiment, the microfluidic elements of the network 105 are arranged within the network portions 110, 120 in a plurality of separable functional modules, whereby the individual functional modules are each marked by outlining rectangular boxes of dot-dashed lines in the illustration shown in this case. Specifically, in this exemplary embodiment the network portion 110 for purifying sample material comprises a pump module 230 for transporting fluids and sample material dissolved therein within the network 105 or between the different functional modules. By way of example only, the pump module comprises two active microfluidic pump chambers 151, 152 and a microfluidic valve 136 for this purpose. In addition, the first network portion 110 comprises a first functional module 250 for providing liquid reagents. By way of example only, arranged in this first functional module are only three pre-storage chambers 161, 162, 163 for a long-term stable pre-storage of liquid reagents. By way of example only, the liquid reagents are aqueous solutions, e.g., buffer solutions for processing a sample substance or components thereof. In another exemplary embodiment, mineral oils, silicone oils, or fluorinated hydrocarbons can also be stored in the pre-storage chambers. In this exemplary embodiment, each pre-storage chamber 161, 162, 163 is fluidically connected to a respective microfluidic valve 131, 132, 133, which in turn are fluidically connected to each other via a first duct interface 255 as well as to a second duct interface 257. By closing the valves 131, 132, 133, the first functional module 250 is separable from the remaining microfluidic network 105. Via the second duct interface 257, the first functional module 250 in this exemplary embodiment is connected to the pump module 230, to a second functional module 261 for adding sample substances, and, by way of example only, to an additional second functional module 262 for adding sample substances. In this case, the second functional module 261 comprises a sample input chamber 167 with a deaeration device 191. The sample input chamber 167 is designed, by way of example, to receive a swab sample, that is to say a sampling device with attached sample material, and is fluidically connected to two valves 134, 135 within the second functional module. Similarly, the additional second functional module 262 comprises a sample input chamber 168 having a deaeration device 192 fluidically connected to two valves 137, 138. In this exemplary embodiment, the sample input chamber 168 is in this case designed, by way of example only, to add a liquid sample. The second functional module 261 and the additional second functional module 262 are fluidically connected to the second duct interface via the valves 134, 137. Via the valves 135, 138, the second functional module 261 and the additional second functional module 262 are in turn fluidically connected to a third duct interface 165, via which there is a fluid connection to the pump module 230 and to a third functional module 270 for filtering the sample material, by way of example only. By closing the valves 134, 135, the second functional module 261 is separable from the remaining microfluidic network 105, just as the additional second functional module 262 is separable from the remaining microfluidic network 105 by closing the valves 137, 138. In this exemplary embodiment, the third functional module 270 comprises, in addition to four microfluidic valves 139, 140, 141, 142, a filter element 170, which is an exemplary silica filter, and which is designed to filter species out of a sample substance, by way of example only. Closing the valves 139, 140, which are fluidically connected to the third duct interface 265 and a fourth functional module 280, in this case causes a fluid flow to be directed across the filter element 170. By closing the valves 139, 141, the third functional module 270 is separable from the functional modules 250, 261, 262 and the pump module 230, and by closing the valves 140, 142, the third functional module 270 is separable from the fourth functional module 280. The fourth functional module 280 in this exemplary embodiment is designed to store a fluid used in the network 105. For this purpose, the fourth functional module 280 comprises, by way of example only, a liquid storage chamber 180 having a deaeration device 193 and two valves 143, 144. In another exemplary embodiment, the fourth functional module 280 can additionally be designed to output sample material. In this exemplary embodiment, the fourth functional module 280 is fluidically connected to the third functional module 270 and the connection duct 125 via a fourth duct interface 285. The valve 144 is in this case designed as a shutoff valve, by way of example only, and is arranged on the connection duct 125. By closing the valve 144, the entire first network portion 110 is therefore separable from the remaining microfluidic network 105. In this exemplary embodiment, the connection duct 125 represents a fluidic connection between the fourth functional module 280 of the first network portion 110 and a first functional module 290 of the second network portion 120 for amplification of sample material. The first functional module 290 of the second network portion 120 is designed in this exemplary embodiment to amplify sample material and comprises, by way of example only, three active microfluidic pump chambers 153, 154, 155 arranged in a row, which, by way of example only, are bracketed by two microfluidic valves 145, 146. The valve 145 in this exemplary embodiment is in the present case arranged directly on the connection duct 125 and designed as a shutoff valve. By closing the valve 145, the entire second network portion 120 is therefore separable from the remaining microfluidic network 105. In this exemplary embodiment, the second network portion 120 also comprises a second functional module 295 having four valves 147, 148, 149, 150, an active microfluidic pump chamber 156, a passive microfluidic chamber 160, and a deaeration device 194. The first functional module 290 and the second functional module 295 are fluidically connected to each other via the valves 146, 147. Separation of the two functional modules 290, 295 from each other is thus possible by way of the valve 146 of the first functional module 290 as well as by means of the valve 147 of the second functional module 295. The second functional module 295 of the second network portion 120 is provided, by way of example only, to provide at least one amplification reaction bead. By way of example only, the amplification reaction bead is a lyophilized or freeze-dried reagent used for producing an amplification reaction mix.

FIG. 3 shows a schematic view of an exemplary embodiment of a microfluidic device 100. The device 100 shown in this case corresponds to or resembles the device described in the preceding drawings. In the illustration shown in this case, the functional modules 250, 261, 262, 270, 280, 290, 295 as well as the pump module 230 are shown in a simplified manner without the individual microfluidic elements. The functional modules 250, 261, 262, 270, 280, 290, 295 and the pump module 230 are arranged in a linear topology. Furthermore, the functional modules 250, 261, 262, 270, 280 as well as the pump module 230 are designed as components of the first network portion 110, and the functional modules 290, 295 as components of the second network portion 120, whereby the first network portion 110 and the second network portion 120 are connected to each other via exactly one connection duct 125. Due to the selected topology, it is only by way of example possible to reduce the size of the dead volumes, reduce the number of pumping steps, and to prevent undesirable microfluidic crosstalk between different process steps of a process.

FIG. 4 shows a schematic illustration of an exemplary embodiment of a microfluidic device 100. The device 100 shown in this case corresponds to or resembles the device with the described network which is described in the preceding drawings. Furthermore, in addition to the microfluidic elements forming the network 105 of the microfluidic device 100, the control ducts used to control the active microfluidic valves 131, 132, 133, 134, 135, 136, 137, 138, 139, 140, 141, 142, 143, 144, 145, 146, 147, 148, 149, 150 and pump chambers 151, 152, 153, 154, 155, 156 are also shown as dashed lines. In this particularly advantageous embodiment, control of these 26 active microfluidic elements 131, 132, 133, 134, 135, 136, 137, 138, 139, 140, 141, 142, 143, 144, 145, 146, 147, 148, 149, 150, 151, 152, 153, 154, 155, 156 is performed by means of a compact control interface 400. The control interface 400 comprises, by way of example only, 20 control connections 401, 402, 403, 404, 405, 406, 407, 408, 409, 410, 411, 412, 413, 414, 415, 416, 417, 418, 419, 420, which can also be referred to as control ports. The control ports in this exemplary embodiment are pneumatic connections, via which an actuation of the active microfluidic elements 131, 132, 133, 134, 135, 136, 137, 138, 139, 140, 141, 142, 143, 144, 145, 146, 147, 148, 149, 150, 151, 152, 153, 154, 155, 156 occurs, by way of example only, by applying positive or negative pressure. As a result, a total of six-fold multiplexing is achieved, as results from the difference in the number of 26 elements and 20 ports. In the drawing shown in this case, the associated six branches of pneumatic control ducts, which enable multiplexing, are illustrated in the form of black nodes. The branching of the control ducts occurs either directly at a control connection or at a suitable point of a control duct. In the exemplary embodiment shown, a branching takes place directly at the control connection 409, by way of example only. The control connection 409 is designed, by way of example only, to collectively control, through a first control duct 431, the valve 141 of the third functional module 270 of the first network portion 110 and, through a second control duct 432, the valve 148 of the second functional module 295 of the second network portion 120. Similarly, in this exemplary embodiment, the first shutoff valve 144 and the second shutoff valve 145 can be controlled by means of a valve control connection 418 for applying pressure. As a result, opening or closing of the connection duct 125 using the valve control connection 418 is possible and the individual network portions 110, 120 are separable from each another. Conversely, a second control connection 410 in this exemplary embodiment has a first branch 441 and a second branch 442, and is thus designed to collectively control both the valve 131 of the first functional module 250 of the first network portion 110 and the valves 149, 150 of the second functional module 295 of the second network portion 120. The control interface 400 also comprises a further control connection 401 for applying pressure, whereby the further control connection 401 in this embodiment is connected to the valve 132 of the first functional module 250 of the first network portion 110 by a further control duct 451. By way of example only, a second further control connection 402 is connected to the pump chamber 152 of the pump module 230 of the first network portion 110, a third further control connection 403 is connected to the valve 137 of the additional second functional module 262 of the first network portion 110, a fourth further control connection 404 is connected to the valve 138 of the additional second functional module 262 of the first network portion 110, a fifth further control connection 405 is connected to the valves 139, 140 of the third functional module 270 of the first network portion 110, a sixth further control connection 406 is connected to the valve 143 of the fourth functional module 280 of the first network portion 110, an eighth further control connection 408 is connected to the valve 142 of the third functional module 270 of the first network portion 110, an 11th further control connection 411 is connected to the valve 133 of the first functional module 250 of the first network portion 110, a 12th further control connection 412 is connected to the valves 134, 135 of the second functional module 261 of the first network portion 110, a 13th further control connection 413 is connected to the pump chamber 151 of the pump module 230 of the first network portion 110, and a 14th control connection 414 is connected to the valve 136 of the pump module 230 of the first network portion 110. Similarly, elements of the second network portion 120 of the device 100 in this embodiment can also be controlled by means of the control interface 400. By way of example only, a seventh further control connection 407 is thus connected to the pump chamber 153 of the first functional module 290 of the second network portion 120, a 15th further control connection 415 is connected to the valve 146 of the first functional module 290 of the second network portion 120, a 16th further control connection 416 is connected to the pump chamber 155 of the first functional module 290 of the second network portion 120, a 17th further control connection 417 is connected to the pump chamber 154 of the first functional module 290 of the second network portion 120, a 19th further control connection 419 is connected to the valve 147 of the second functional module 295 of the second network portion 120, and a 20th further control connection 420 is connected to the pump chamber 156 of the second functional module 295 of the second network portion 120.

FIG. 5 shows a schematic view of an exemplary embodiment of a microfluidic device 100. The device 100 shown in this case corresponds to or resembles the device described in the preceding drawings. For illustration, in the drawing shown in this case, the microfluidic network 105 is shown in a simplified manner. In this exemplary embodiment, the microfluidic device 100 comprises two network portions 110, 120 which are connected to each other via a microfluidic connection duct 125 and a pneumatic control interface 400 having four control connections 409, 410, 418, 500. In this exemplary embodiment, the control connections 409, 410, 418, 500 are each designed to control microfluidic elements of both the first network portion 110 and the second network portion 120. As a result of this four-fold multiplexing, a total of eight active microfluidic elements of the device 100 can be controlled in an advantageous manner by means of the four control connections, four of them per network portion 110, 120. The multiplexing is achieved by branching the control ducts at a total of four node points 409, 418, 441, 505.

FIG. 6 shows a perspective top view of an exemplary embodiment of a microfluidic device 100. The device 100 shown in this case corresponds to or resembles the device described in the preceding drawings. In this exemplary embodiment, the device 100 comprises a reagent bar 600 arranged within the three pre-storage chambers 161, 162, 163 having three compartments in each of which different liquid reagents can be pre-stored with long-term stability. Correspondingly, the pre-storage chambers 161, 162, 163 can also be referred to as reagent pre-storage chambers. In addition, within the sample input chamber 167 of the second functional module described in the preceding FIGS. 2 to 4, a sampling device 605 is arranged, by way of example only, to enable transfer of a swab sample thereon into the network 105 of the microfluidic device 100.

FIG. 7 shows a top view of an exemplary embodiment of a microfluidic device 100. The device 100 shown in this case corresponds to or resembles the device described in the preceding drawings. In the illustration shown in this case, some of the elements of the microfluidic device 100 described in the preceding drawings are numbered by way of example. Specifically, the microfluidic valve 146, the microfluidic pump chambers 151, 152, 153, 154, 155, 156, the microfluidic chamber 160, the three pre-storage chambers 161, 162, 163, the two sample input chambers 167, 168, the microfluidic filter element 170, and the liquid storage chamber 180 are numbered. Furthermore, the first network portion 110 for purification of a sample substance, the second subnetwork 120 for amplification of sample material, and the pneumatic control interface 400 with an exemplarily numbered further control connection 401 are indicated by dashed lines. By way of example only, the device has an overall lateral dimension of 118×78 mm². In a further embodiment, the overall dimension can be 50×25 mm² to 300×200 mm², preferably 75×25 mm² to 200×100 mm². In this exemplary embodiment, the fluidic and pneumatic microducts also have a cross-sectional dimension of only 600×400 μm², by way of example only. In a further exemplary embodiment, the cross-sectional dimensions of the fluidic and pneumatic microducts can be 100×100 μm² to 3×3 mm², preferably 300×300 μm² to 1×1 mm². The effective displacement volume of a microfluidic pump chamber in this exemplary embodiment is 20 μL, the effective displacement volume of a microfluidic valve is only 125 nL, and the volume of the microfluidic chamber with filter element in this exemplary embodiment is only 8 μL. In other exemplary embodiments, the effective displacement volume of a microfluidic pump chamber can be from 1 μL to 50 μL, preferably from 5 μL to 30 μL, the effective displacement volume of a microfluidic valve can be 50 nL to 1 μL, preferably 100 nL to 300 nL, and the volume of the microfluidic chamber with filter element can be 3 μL to 20 μL, preferably 5 μL to 10 μL. In this exemplary embodiment, for microfluidic processing within the device 100, a pressure difference (positive pressure or negative pressure relative to atmospheric pressure), which is used to generate the microfluidic flow by pressuring or aspirating by means of a pump chamber or controlling the microfluidic flow by means of a valve, of 700 mbar is applicable, by way of example only. In a further exemplary embodiment, the pressure difference can be 100 mbar to 2000 mbar, preferably 400 mbar to 1500 mbar.

FIG. 8 shows a perspective top view of a section of the microfluidic device 100 according to an exemplary embodiment. The device 100 shown in this case corresponds to or resembles the device described in the preceding drawings. In the drawing shown in this case, by way of example, the microfluidic valve 131, the microfluidic pump chambers 151, 152, the sample input chamber 168, the microfluidic chamber having the filter element 170, the deaeration device 193, and the pneumatic control connections 405, 409, 410 are numbered. The microfluidic device 100 in this exemplary embodiment is realized by two rigid polymeric components, 800, 805, connected to each other via a flexible membrane 810. The lower polymer component 800 is terminated by another polymer film to seal the microducts present in the polymer component. The multi-layer construction thus enables in particular the realization of fluidic and pneumatic microducts on at least two different lateral planes. In this way, fluidic and pneumatic microducts are crossable and a particularly high degree of integration of the microfluidic device 100 can be achieved with a central and particularly compact configuration of the pneumatic control interface described in FIG. 4. By applying positive or negative pressure to the pneumatic control connections, the flexible polymer membrane can be deflected in recesses present on the microfluidic valves and pump chambers via the pneumatic microducts in order to achieve the opening or closing of a microfluidic valve or a pump chamber. By pumping out or expelling liquid by means of a pump chamber, the microfluidic flow within the network 105 can be precisely controlled when the valve is in a suitable position. In other exemplary embodiments, e.g., the following materials can be used for the realization of the microfluidic analysis device: Primarily polymers such as polycarbonate (PC), polystyrene (PS), styrene-acrylonitrile copolymer (SAN), polypropylene (PP), polyethylene (PE), cycloolefin copolymer (COP, COC), polymethyl methacrylate (PMMA), polydimethyl siloxane (PDMS) or thermoplastic elastomers (TPE) such as polyurethane (TPU) or styrene block copolymer (TPS). The device can be manufactured, e.g., by series production methods like injection molding, compression molding, thermoforming, punching or laser transmission welding.

FIG. 9 shows a perspective bottom view of a section of the microfluidic device 100 according to an exemplary embodiment. The device 100 shown in this case corresponds to or resembles the device described in the preceding drawings. In the drawing shown in this case, by way of example, the microfluidic valve 131, the microfluidic pump chambers 151, 152, the sample input chambers 167, 168, the microfluidic chamber having the filter element 170, the deaeration device 193, and the pneumatic control port 405 are numbered.

FIG. 10 shows a flow chart of a method 1000 for operating a microfluidic device according to an exemplary embodiment. The device operated using the method can be the device described in the preceding drawings. The method 1000 comprises a step 1005 of extracting sample material using the first network portion. By way of example only, a sample substance is purified in the network portion and sample material, e.g. DNA or RNA, is extracted from the sample substance and transferred to the microfluidic network. This is followed by a step 1010 of transferring sample material from the first network portion to the second network portion, whereby a solution containing previously extracted sample material is transferred from the first network portion to the second network portion. In the following step 1015 of amplification sample material using the second network portion, sample material is amplified in the second network portion and thus made accessible for, e.g., fluorometric verification. In a further exemplary embodiment of the method 1000, the step 1005 of extraction or the steps 1010, 1015 of transfer and amplification can be omitted. For example, the first step 1005 of extraction can be omitted if the species being verified is already present in extracted form in an added sample liquid. The second step 1010 of transfer and the third step 1015 of amplification can be omitted, e.g., if only purification or extraction of sample material is performed in the device and the sample material is removed from the microfluidic device again, e.g. after performing the first step 1005 of extraction.

FIG. 11 is a flow chart of a method 1000 for operating a microfluidic device according to an exemplary embodiment. The method 1000 shown in this case corresponds to or resembles the method described in the preceding FIG. 10, with the difference that the individual steps 1005, 1010, 1015 have additional substeps. In this exemplary embodiment, the step 1005 of extraction comprises a substep 1105 of mixing, and a substep 1110 of dissolving, and a substep 1115 of lysis, and a substep 1120 of filtering, and a substep 1125 of rinsing. In this exemplary embodiment, the step 1010 of transfer comprises a substep 1130 of elution and the step 1015 of amplification comprises a substep 1135 of dissolving, and a substep 1140 of reproduction, and a substep 1145 of acquisition. In the substep 1105, mixing of a liquid sample added into the sample input chamber is performed, only by way of example, using a binding buffer. The mixing is performed in particular using the first functional module, the additional second functional module, the pump module and a plurality of the following elements: microfluidic valves, microfluidic pump chambers, a sample input chamber and a deaeration device. In this exemplary embodiment, the binding buffer is upstream in exactly one of the three pre-storage chambers of the first network portion. In this exemplary embodiment, the adjacent functional modules comprise microfluidic valves, which are closed in this substep 1105, such that undesirable microfluidic crosstalk with the adjacent functional modules is prevented.

In the substep 1110 of dissolving, in this exemplary embodiment sample material is dissolved within the sample input chamber, which is initially present and attached to a sampling device added to the sample input chamber. Dissolving is performed, by way of example only, by means of a transfer liquid introduced into the chamber, in particular using the first and second functional modules and the pump module and a plurality of the following elements: microfluidic valves, microfluidic pump chambers, pre-storage chambers, a sample input chamber and a deaeration device. The transfer liquid is upstream in precisely one of the pre-storage chambers, by way of example only. The adjacent functional modules comprise in particular microfluidic valves, which are closed in this substep 1110 such that undesirable microfluidic crosstalk with the adjacent functional modules is prevented.

In the substep 1115 of lysis, in this embodiment a lysis buffer is added to a sample substance added to at least one of the sample introduction chambers, and the sample substance or components thereof are tempered to effect thermally induced lysis of components of the sample substance. The components of the sample substance to be lysed in this substep 1115 are bacteria, by way of example only. In another exemplary embodiment, e.g., these can be other cells. Lysis is performed in particular using the first, second and additional second functional modules and the pump module, as well as a plurality of the following elements: microfluidic valves, microfluidic pump chambers, pre-storage chambers, a sample input chamber and a deaeration device. The lysis buffer is upstream in precisely one of the pre-storage chambers, by way of example only. The adjacent functional modules comprise in particular microfluidic valves, which are closed in this substep 1115, such that undesirable microfluidic crosstalk with the adjacent functional modules is prevented. In an alternative exemplary embodiment of the substep 1115 of lysis, thermally induced lysis of components of the sample substance can be performed by tempering the filter element after the components of the sample substance have been enriched on the filter element, during the substep 1120 of filtering.

During the substep 1120 of filtering, in this exemplary embodiment, a filtering and binding of components of the sample substance added to at least one of the sample input chambers is performed using the filter element such that the components of the sample substance are enriched on the filter element. The filtering is performed in particular using the third, fourth, second or additional second functional module and pump module, as well as a plurality of the following elements: microfluidic valves, microfluidic pump chambers, a sample input chamber, the filter element, the liquid storage chamber, and deaeration devices. The adjacent functional modules comprise in particular microfluidic valves, which are closed in this substep 1120 such that undesirable microfluidic crosstalk with the adjacent functional modules in this substep is prevented. For example, a binding buffer with sample material from one of the sample input chambers is pumped by means of the filter element into the liquid storage chamber, whereby components of the sample material, for example DNA or RNA, are attached and concentrated on the filter element. The substep 1120 of filtering is performed in this exemplary embodiment after a performance of the substep 1105 of mixing, the substep 1110 of dissolving and the substep 1115 of lysis. In another exemplary embodiment, the substep 1120 of filtering can occur prior to or after the substep of lysis depending upon the exact embodiment of the method: If lysis occurs in one of the sample input chamber, then the substep of filtering can be performed after the substep of lysis. If lysis occurs on the filter element, on the other hand, the substep of filtering can be performed prior to the substep 716 of lysis.

In the substep 1125 of rinsing, in this exemplary embodiment, the microfluidic network is rinsed with a wash buffer, e.g. to eliminate residues of the binding buffer present in the microfluidic network after the substep 1120 of filtering. Rinsing is performed in particular using the first, third, and fourth functional modules, as well as the pump module and a plurality of the following elements: microfluidic valves, microfluidic pump chambers, pre-storage chambers, the liquid storage chamber, and a deaeration device. The wash buffer is upstream in precisely one of the pre-storage chambers, by way of example only.

In the substep 1130 of elution, in this exemplary embodiment, sample material present on the filter element is dissolved in order to make it accessible for molecular diagnostics verification by means of an amplification reaction. The sample material is DNA, by way of example only, in another example, it can also be RNA. To this end, an elution buffer is pumped over the filter element to transfer sample material present on the filter element to the elution buffer, by way of example only. In connection with the elution, in this embodiment, the elution buffer is also transferred with sample material from the first network portion to the second network portion via the microfluidic connection duct. The elution is performed in particular using the first, third, and fourth functional modules of the first network portion, as well as the first functional module of the second network portion and the pump module as well as a plurality of the following elements: microfluidic valves, microfluidic pump chambers, microfluidic connection duct, pre-storage chambers, liquid storage chamber, and a deaeration device. The elution buffer is upstream in precisely one of the pre-storage chambers, by way of example only.

In the substep 1135 of dissolving, in this exemplary embodiment the elution buffer is employed with the sample material contained in order to dissolve a freeze-dried or lyophilized reagent, which can also be referred to as reaction bead, and in this way provide a reaction mix for a subsequent amplification reaction. By way of example only, this is a master mix for performing a polymerase chain reaction. Dissolving is performed in particular using the first and second functional module of the second network portion as well as a plurality of the following elements: microfluidic valves, microfluidic pump chambers, microfluidic chamber, and the deaeration device. In this exemplary embodiment, the freeze-dried or lyophilized reagent or reaction bead is present in a pump chamber or the passive microfluidic chamber. Pumping of the elution buffer with sample material into at least one of the elements for dissolving the reaction bead is performed in this exemplary embodiment using pump chambers. After releasing the reaction bead, by way of example only, the resulting reaction mix is transferred from the second functional module of the second network portion to the first functional module of the second network portion.

In the substep 1140 of reproduction, in this exemplary embodiment at least one amplification reaction is performed in order to amplify sample material present in the reaction mix. Reproduction is performed in particular using the first functional module of the second network portion as well as a plurality of the following elements: microfluidic valves, microfluidic pump chambers. Reproduction is performed, by way of example only, by a polymerase chain reaction. In another exemplary embodiment, the reproduction can also be performed by an isothermal amplification method. As part of the substep of reproduction, the microfluidic pump chambers in which the reaction mix is present can therefore also be tempered in order to create suitable physical conditions that enable the amplification reaction to take place.

In the substep 1145 of acquisition, in this exemplary embodiment a signal is acquired in order to verify the occurrence of at least one amplification reaction as part of the substep 1140 of reproduction. The signal is, by way of example only, an optical signal, e.g., a fluorescence signal emanating from at least one fluorescent dye indicative of the occurrence of at least one amplification reaction. In another exemplary embodiment, the substep 1145 of acquisition can be performed at the same time as the substep 1140 of reproduction, or after the substep 1140 of reproduction. Optionally, during the substep 1145 of acquisition, output of the signal or a variable derived therefrom is also performed to output the result of the sample analysis performed in the microfluidic analysis device to a user.

In further embodiments of the method, individual substeps can be omitted or performed repeatedly, or can be swapped in order with other substeps as exemplified in the following exemplary embodiments.

FIG. 12 shows a flow chart of an exemplary embodiment of a method 1000 for operating a microfluidic device. The method 1000 shown in this case corresponds to or resembles the method described in the previous FIGS. 10 and 11. In this variant of the method 1000, the substeps 1105 of mixing, 1115 of lysis, 1120 of filtering, 1125 of rinsing, 1130 of elution, 1135 of dissolving, 1140 of reproduction, and 1145 of acquisition are performed successively.

FIG. 13 shows a flow chart of a method 1000 for operating a microfluidic device according to an exemplary embodiment. The method 1000 shown in this case corresponds to or resembles the method described in the previous FIGS. 10, 11, and 12. In this variant of the method 1000, the substeps 1110 of dissolving, 1120 of the filtering, 1115 of lysis, 1125 of rinsing, 1130 of elution, 1135 of dissolving, 1140 of reproduction, and 1145 of acquisition are performed successively.

FIG. 14 shows schematic view of an exemplary embodiment of an analysis device 1400 for receiving a microfluidic device. The analysis device 1400 is designed, by way of example only, to receive a microfluidic device by means of an input port 1405 as described in the preceding FIGS. 1 to 9 to perform analysis processes within the device. In this exemplary embodiment, the analysis device 1400 comprises a control device 1410 designed to control the method steps described in the preceding FIGS. 10 to 13 with respect to the device.

1. A microfluidic device for analyzing sample material, comprising:
a microfluidic network including a first network portion configured to purify a sample material, and a second network portion configured to amplify the sample material, wherein
the second network portion is connected to the first network portion via a connection duct, and
the first and second network portions are configured to be separable from one another.

2. The microfluidic device according to claim 1, wherein the first network portion and the second network portion are arranged in a linear topology.

3. The microfluidic device according to claim 1, wherein the first network portion and/or the second network portion comprises a plurality of fluidically separable functional modules.

4. The microfluidic device according to claim 3, wherein:
the device comprises a control connection for applying pressure; and
the control connection is connected via a first control duct to an element of a functional module of the first network portion and via a second control duct to an element of the functional module of the second network portion.

5. The microfluidic device according to claim 1, wherein;
the first network portion comprises a first network functional module for providing liquid reagents, and/or a second first network functional module for adding the sample material, and/or a third first network functional module for filtering the sample material, and/or a fourth first network functional module for storing a liquid and/or a pump module for establishing a fluid transport between the functional modules; and/or
the second network portion comprises at least a first second network functional module for amplifying the sample material and/or a second network functional module for releasing at least one amplification reaction bead.

6. The microfluidic device according to claim 5, wherein:
the first network portion includes the fourth first network functional module;
the second network portion includes the first second network functional module; and
the fourth first network functional module of the first network portion and the first second network functional module of the second network portion are fluidically connected via the connection duct.

7. The microfluidic device according to claim 6, wherein the fourth first network functional module of the first network portion comprises a first shutoff valve configured to close the connection duct, and/or the first second network functional module of the second network portion comprises a second shutoff valve configured to close the connection duct.

8. The microfluidic device according to claim 7, further comprising:
a valve control connection for applying pressure, wherein at least one of the first shutoff valve and the second shutoff valve are controlled by the valve control connection.

9. The microfluidic device according to claim 5, wherein;
the first network portion includes the fourth first network functional module; and
the fourth first network functional module of the first network portion is designed to output a liquid with the sample material.

10. The microfluidic device according to claim 5, wherein the first network portion comprises an additional second functional module configured to add the sample.

11. The microfluidic device according to claim 4, wherein:
the device comprises at least one further control connection for applying pressure, and
the further control connection is connected via a further control duct to either an element of a functional module of the first network portion or to an element of a functional module of the second network portion.

12. A method for operating a microfluidic device according to claim 1, the method comprising:
extracting the sample material using the first network portion;
transferring the sample material from the first network portion to the second network portion; and
amplifying the sample material using the second network portion.

13. The method according to claim 12, wherein:
the extracting comprises mixing and/or dissolving and/or lysing and/or a filtering and/or rinsing; and/or
transferring comprises eluting; and/or
amplifying comprises dissolving, and/or reproducing, and/or acquiring.

14. A control device designed to perform and/or control the method according to claim 12 in corresponding units.

15. A computer program designed to perform and/or control the method according to claim 12.
